(19) United States
(12) Patent Application Publication
(10) Pub. No.: US 2007/0087055 A1
(43) Pub. Date: Apr. 19, 2007

(54) Directly compressible extended release alprazolam formulation

(76) Inventors: David Jan, Parkland, FL (US); Xiu Xiu Cheng, Weston, FL (US); Guohua Zhang, Parsippany, NJ (US); Roger Carter, Miami, FL (US)

Correspondence Address: HEDMAN & COSTIGAN P.C., 1185 AVENUE OF THE AMERICAS, NEW YORK, NY, 10036, US

(21) Appl. No.: 11/250,640

(22) Filed: Oct. 14, 2005

(51) Int. Cl. A61K 31/5513; A61K 9/22

(52) U.S. Cl. 424/468; 514/221

(57) ABSTRACT

A directly compressible extended release pharmaceutical dosage form comprising alprazolam and at least two high viscosity polymers, wherein said first high viscosity polymer and said second high viscosity polymer are present in a ratio from about 4:1 to about 2:1.

### BACKGROUND OF THE INVENTION

The present invention relates to a unit dose formulation wherein the active ingredient is mixed with a two high viscosity polymers. More specifically, the present invention relates to an oral dosage form comprising a water insoluble drug, preferably an anxiolytic drug, or a benzodiazepine such as alprazolam, or a pharmaceutically acceptable salt thereof, such as those described in U.S. Pat. No. 3,987,052 (which is incorporated herein by reference).

The drug alprazolam is commercially available in immediate release form in 0.25 mg, 0.5 mg, 1 mg and 2 mg tablets under the tradename XANAX® and in extended release form in 0.5 mg, 1 mg, 2 mg and 3 mg tablets under the tradename XANAX® XR from Pharmacia & Upjohn Company. See Physicians' Desk Reference, pp. 2763-71 (59^{th} Ed. 2005) (which is incorporated herein by reference).

Alprazolam is useful in the treatment of central nervous system conditions and disorders including general anxiety disorder, anxiety associated with depression, panic disorder and panic attacks.

Alprazolam is a member of the 1,4-benzodiazepine class of central nervous system (CNS)-active compounds and is an effective anxiolytic and anti-panic agent. Immediate-release versions of the alprazolam product may be prescribed for administration of up to four times daily for treatment of anxiety, and sometimes over four doses per day for treatment of panic disorder. This more than once-a-day dosing results in major problems with patient compliance. Furthermore, the problem of breakthrough anxiety is always prevalent with repeated daily dosing. Therefore, extended release formulations of alprazolam are advantageous to reduce the frequency of dosing, to provide more uniform blood levels of the drug for continuous control of symptoms, for greater ease of discontinuation and for greater patient compliance.

Extended release alprazolam formulations have been described, including formulations wherein alprazolam is dispersed in a polymer matrix, for example a hydroxypropyl methylcellulose (HPMC) matrix. Franz et al. (1987), Journal of Controlled Release 5, 159-172, which examined the effects of several formulation variables on in vitro alprazolam release rate from matrix formulations comprising hydroxypropyl methylcelluloses (HPMCs) of different viscosity grades, sodium carboxymethylcellulose (sodium CMC) and lactose. These variables included ratio of high to low viscosity HPMC, ratio of sodium CMC to lactose and matrix drug loading. Franz et al. demonstrated that while differing proportions of polymers may be employed to produce an extended release of the active, the exact release rate cannot be predicted simply by the choice and/or ratio of polymer.

Additionally, as described above, the commercially available XANAX® XR formulation is an extended release dosage form. This product comprises alprazolam as the active ingredient in combination with various excipients, two of which are a high viscosity polymer and a low viscosity polymer. This formulation is described in U.S. patent application Ser. No. 10/464045 (Publication No. 2004/0006072). The application describes an extended release alprazolam dosage form that employs a relatively high viscosity HPMC and a relatively low viscosity HPMC. Specifically, the application teaches the use a combination of low viscosity HPMC and high viscosity HPMC at a ratio of about 40:60 to about 60:40. This results in about 110 mg to about 135 mg per tablet of polymer.

Despite these prior art teachings, there still exists a need in the art for a low cost easily producible extended release alprazolam dosage form.

Accordingly, a novel aspect of the present invention involves producing an extended release of active using more than one high viscosity polymer in a directly compressible dosage form.

It is a further object of the present invention to provide a highly bioavailable dosage form in a simple and inexpensive manner.

It is also an object of the present invention to provide a directly compressible extended release dosage form comprising two high viscosity polymers in a ratio from about 4:1 to about 2:1.

It is also an object of the present invention to provide a directly compressible extended release dosage form comprising a total amount of polymer that is about 18% to about 25% of the total tablet weight.

Other objects, features and advantages of the invention are not taught in the prior art but will be more apparent to those versed in the art from the following specification, taken in conjunction with the accompanying claims.

### SUMMARY OF THE INVENTION

The foregoing objectives are met by a directly compressible oral dosage form comprising:

(a) a water insoluble drug;

(b) a filler;

(c) a first high viscosity polymer;

(d) a second high viscosity polymer;

(e) optionally a glidant; and

(f) optionally a lubricant;

wherein said first high viscosity polymer and said second high viscosity polymer are present in a ratio of from about 4:1 to about 2:1.

In one preferred embodiment of the present invention, the objectives are met by a directly compressible oral dosage form comprising:

(a) a slightly soluble to insoluble benzodiazepine drug;

(b) a filler;

(c) a first high viscosity polymer;

(d) a second high viscosity polymer;

(e) optionally a glidant; and

(f) optionally a lubricant;

wherein said first high viscosity polymer and said second high viscosity polymer are present in a ratio of from about 4:1 to about 2:1.

The present invention uses a novel combination of at least two high viscosity polymers to produce an extended release of active from a directly compressible dosage formulation. Specifically, the present invention employs the use of two high viscosity hypromellose polymers, more specifically types 2208 and 2910. The inventors of the present invention surprisingly have found that the use of two high viscosity polymers in a ratio of from about 4:1 to about 2:1 and wherein the total weight of the high viscosity polymers is from about 18% to about 25% of the total weight of the final tablet, results in a highly desirable extended release alprazolam product. In a preferred embodiment, hypromellose 2910 and 2208 had an absolute viscosity of about 4000 cPs (centipoises), while a preferred hypromellose had an absolute viscosity of about 10,000 cPs. In contrast to U.S. patent application Ser. No. 10/464,045 (“the '045 application”), the present invention only uses about 62 mg to about 86 mg total hypromellose per tablet (with a theoretical tablet weigh of 345 mg). The '045 application characterizes hypromellose with viscosity ranges from 3000-5600 cPs as “high viscosity.” The '045 application characterizes viscosity ranges from 80-120 cPs as low viscosity. Further, the '045 application employs two METHOCEL® K grades (type 2208) whereas the present invention employs one METHOCEL® K grade and one METHOCEL® E grade (type 2910). The “E” grade typically hydrates faster then the “K” grade, and the “K” grade typically erodes faster. The “K” and “E” grade differ in methoxy content and hydroxypropyl content. The differences in methoxy content and hydroxypropyl content provide each type of hypromellose with their distinct erosion and hydration characteristics. Therefore, substitution of an “E” grade hypromellose for a “K” grade hypromellose is novel in pharmaceutical formulations because of these differences in erosion and hydration characteristics. Moreover, the particular combination, from about 4:1 to about 2:1 of these two types of high viscosity polymers produces an unexpectedly desirable extended release oral dosage formulation. The present invention as described herein produced a uniform and extended release of alprazolam from a direct compression matrix comprising the two high viscosity polymers. Furthermore, there is a great advantage in cost in the simple use of two high viscosity polymers in a blended directly compressible dosage formulation. The procedure is simple, involves less steps than complicated wet granulation, mircroencapsulation, multiple coating systems and/or multiple pellet (with or without multiple coating) systems. This novel approach of dry blending the components in combination with direct compression to produce an extended release dosage form unexpectedly yields an improved, cost effective and highly bioavailable product. Additionally, the use of extended release products also greatly improves patient compliance.

### BRIEF DESCRIPTION OF THE DRAWINGS

FIG. 1 is a graph depicting the linear plot of the mean plasma alprazolam concentration versus time of the formulation described in Example 1 and the commercially available extended release alprazolam product (XANAX® XR) under fasting conditions with N=36.

### DETAILED DESCRIPTION OF THE INVENTION

Benzodiazepines comprise alprazolam, bromazepam, chlordiazepoxide, clobazam, clonazepam, clorazepate, diazepam, flunitrazepam, flurazepam, loprazolam, lormetazpam, nitrazepam, oxazepam, and tamazepam.

Alprazolam is a preferred drug for use in the present invention. The alprazolam can be in various forms, such as uncharged molecules, molecular complexes, pharmacologically acceptable salts such as hydrochloride, hydrobromide, sulfate, laurate, palmitate, tartrate, oleate, phosphate, nitrite, borate, acetate, maleate and salicylate. In embodiments comprising acidic drugs, salts of metals, amines or organic cations; for example, quaternary ammonium can be used. Derivatives of drugs such as ester, ethers and amides also can be used. Additionally, a drug that is water insoluble can be used in a form that is a water soluble derivative thereof to serve as a solute, and on its release from tablet, is converted by enzymes, hydrolyzed by body pH or other metabolic processes to the original biologically active form.

In a preferred embodiment, alprazolam is blended (in one or more steps) with at least one filler, at least two high viscosity polymers and optionally a glidant. The blend then is mixed further with a lubricant in preparation for compression into a tablet. The preferred composition of the present invention is given below:

| | Preferred | Most Preferred |
|---|---|---|
| drug | 0.01-5% | 0.1-2.5% |
| filler | 50-99% | 65-85% |
| first polymer | 1-30% | 2-25% |
| second polymer | 1-25% | 2-20% |
| glidant | 0-5.0% | 0.1-2.0% |
| lubricant | 0-10% | 0.10-3.0% |

The pharmaceutical formulations of the present invention may contain one or more fillers for use in the granulation and/or direct compression. Pharmaceutically acceptable fillers are added to improve the size of a pharmaceutical formulation where the active ingredient is relatively low, i.e. bulking up, and are also used to improve compression and tabletting qualities of the material as is known to those of ordinary skill in the art. In the final tablet formulation of the preferred embodiment, the amount of filler preferably ranges from about 50 to about 99% of the total tablet weight, most preferably from about 65 to about 85% of the total tablet weight. Suitable fillers that may be employed in the present invention include, but are not limited to any conventionally known pharmaceutically acceptable diluent, such as microcrystalline cellulose, lactose, dextrose, sucrose, sodium chloride, maltose, fructose, galactose, gelatin, polyvinylpyrrolidone, rice starch, corn starch, calcium carbonate and the like or mixtures thereof. Lactose monohydrate, which is considered an inert pharmaceutical excipient, may be added as a directly compressible tabletting excipient. Lactose monohydrate also is used as a filler to achieve content uniformity of the finely divided active ingredients. The preferred filler is lactose monohydrate.

Polymers are employed in the present invention to act as extended release controlling agents and as binders. Suitable polymers that may be employed in the present invention are a combination of at least two high viscosity polymers. Suitable high viscosity polymers include, but are not limited to, hypromellose available from DOW under the tradename METHOCEL®, such as, K4M, K15M, K100MP, E4MP, E10MP CR. The viscosity of the hypromelloses that are useful in the practice of the present invention range from 3,000-120,000 mPa·s. Additional suitable polymers that can be used with the present invention include, high viscosity grades of methylcellulose, hydroxypropyl methylcellulose and hydroxyethyl cellulose, hydroxypropyl cellulose. Further suitable high viscosity polymers are disclosed in the Handbook of Pharmaceutical Excipients, (4^{th} Ed. 2003) and are incorporated herein by reference. The inventors of the present invention have surprisingly found that the use of two high viscosity polymers in a ratio ranging from about 4:1 to about 2:1 and wherein the total weight of the high viscosity polymers is about 18% to about 25% of the total weight of the final tablet, results in a highly desirable extended release alprazolam product that may be prepared by direct compression and successfully manufactured according to cGMP. Numerous attempts were made using compositions comprising Methocel grades K and K; K and E; and E and E. The inventors surprising found a combination of polymers that gave a desired result. Although only two polymers were used, more than two polymers may be used in some embodiments.

Lubricants may be employed in the present invention to aid in tabletting and avoid sticking of the blended material on the metallic surface of the tabletting apparatus. An effective amount of any generally accepted pharmaceutical tabletting lubricant may be added to compress the tablets. If a lubricant is added it may be present in an amount from about 0 to about 10%, preferably from about 0.1 to about 3% by weight of the total tablet. Tablet lubricants are preferably selected from the group consisting of glyceryl monostearates, magnesium stearate, palmitic acid, talc, carnauba wax, calcium stearate, sodium or magnesium lauryl sulfate, calcium soaps, zinc stearate, polyoxyethylene monostearates, calcium silicate, silicon dioxide, hydrogenated vegetable oils and fats, or stearic acid. In the preferred embodiment, magnesium stearate is used in an amount of about 1% of the total tablet weight.

A glidant also may be included in the compositions of the present invention. The glidant can be a fumed colloidal dioxide or some other suitable glidant and preferably a fumed colloidal silicon dioxide such as Cab-O-Sil®. Other known glidants are disclosed in the Handbook of Pharmaceutical Excipients, (4^{th} Ed. 2003) and are incorporated herein by reference. The glidant is preferably used in a concentration of 0 to about 5%, and most preferably at a concentration of from about 0.1 to about 2% of the total tablet weight.

Coloring additives may also be added to the pharmaceutical dosage form of the present invention. Coloring additives are well known in the art and are added for aesthetic purposes as well as to distinguish one dosage strength from another. Coloring additives preferably comprise less than about 1% of the total weight of the tablet and most preferably less than about 0.5% of the total tablet weight.

A tablet disintegrant may be added to the direct compression process for its wicking effect (i.e., the ability of particles to draw water into the porous network of a tablet) and swelling ability. Some of these disintegrants also serve as excellent binders and are able to substantially improve the mechanical strength of the formulation. Suitable disintegrants are carboxymethyl cellulose sodium, carboxymethyl cellulose calcium, crospovidone, sodium starch glycolate, cornstarch, insoluble cationic-exchange resins such as polyacrylin, microcrystalline cellulose and croscarmellose. These may be added in amounts that are conventional in the pharmaceutical formulation arts.

The tablets of the invention may also include a seal or sugar coating layer. The seal or sugar coating influences the tablet moisture, surface roughness, and coating efficacy and uniformity. The seal or sugar coating may be about 1.0-5.0% of the total weight of the tablet.

### DESCRIPTION OF THE PREFERRED EMBODIMENTS

### Example 1

A 3 mg strength alprazolam extended release tablet of is prepared as follows:

A. Blending

TABLE 1

| | | Amount per | Amount per |
|---|---|---|---|
| Ingredients | % | Unit (mg/tab) | Batch (kg) |
| Alprazolam | 0.8696 | 3.00 | 0.450 |
| Lactose Monohydrate | 75.54 | 260.6 | 39.09 |
| Hypromellose 2208 | 16.50 | 56.93 | 8.540 |
| Hypromellose 2910 | 5.501 | 18.93 | 2.847 |
| Colloidal Silicon Dioxide | 0.500 | 1.725 | 0.2588 |
| Magnesium Stearate | 1 | 3.450 | 0.5175 |
| Coloring Additive | 0.1 | 0.3450 | 0.05175 |
| Total | 100.00 | 345.0 | 51.75 |

Mix approximately 1 kg of the lactose monohydrate and 0.450 kg of alprazolam for two minutes. Next add 0.05175 kg of the coloring agent to the above mix and continue mixing for 3 minutes. Then mix approximately 4 kg of lactose monohydrate with the above mixture in a 16 quart V-Blender for fifteen (15) minutes. Next place an additional 34.09 kg of lactose monohydrate, the mixture from above, 8.450 kg of hypromellose 2208, 2.847 kg of hypromellose 2910 and 0.2588 kg of colloidal silicon dioxide into a 5 ft³ V-blender and mix for 15 minutes. Then pass the resulting mixture through a comil equipped with a 20 mesh stainless steel screen with no spacer. Charge the screened material into a 5 ft³ V-blender and mix for twenty (20) minutes. Screen 0.5175 kg of magnesium stearate through a #30 mesh screen then add the screened magnesium stearate to the above blend and blend for an additional five minutes.

B. Compression

The blend is then compressed into tablets with tablet weights around 345 mg on a rotary tablet press or an alternative equipment of the same operation principle.

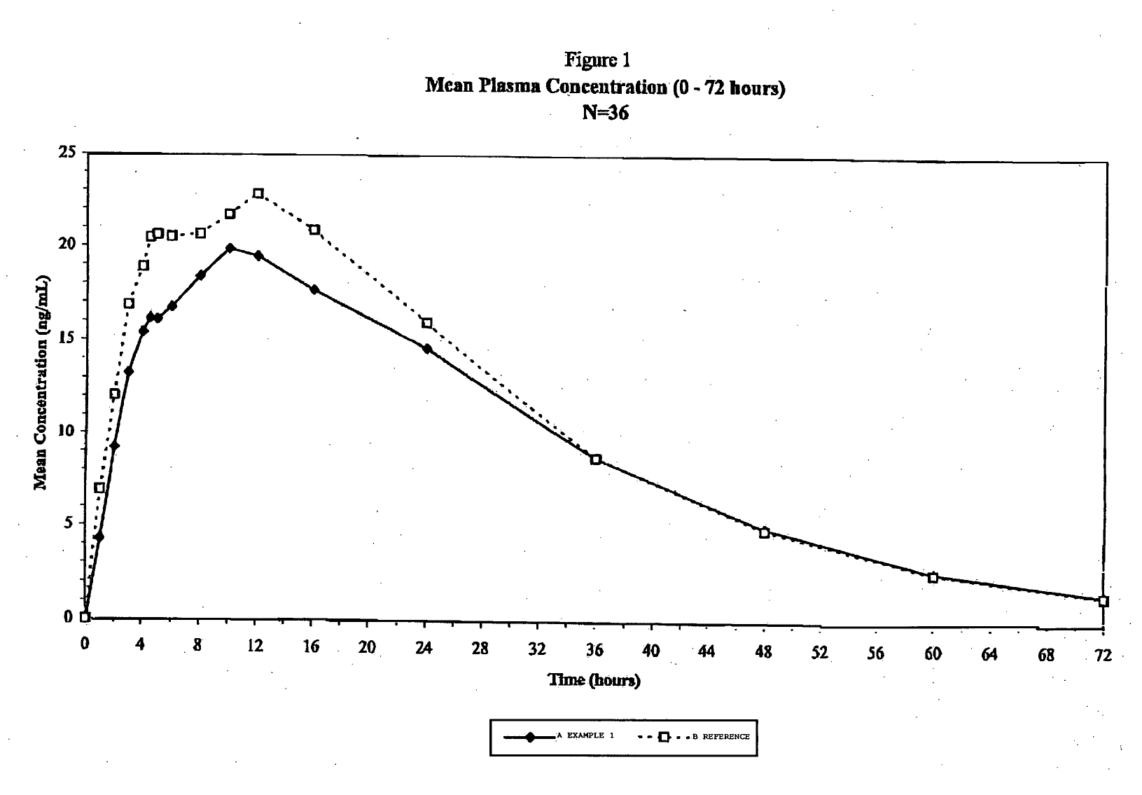

Table 2 is a summary of the bioavailability comparison data under fasting conditions, test/reference ratio, shown in FIG. 1 wherein the test product was prepared according to Example 1 and the XANAX® XR product is the reference product in a two way crossover biostudy with n=36.

TABLE 2

| | | |
|---|---|---|
| Test Mean | Ref Mean | Test/Ref Ratio |

Non-transformed data

| Cₘₐₓ(ng/ml) | 21.06 | 24.91 | 84.55 |
|---|---|---|---|
| AUC₀₋ₜ(ng · hr/ml) | 677.96 | 750.96 | 90.28 |
| AUC_{inf}(ng · hr/ml) | 711.80 | 785.40 | 90.63 |
| Tₘₐₓ(hr) | 10.32 | 10.81 | 95.50 |
| kₑₗᵢₘ | 0.0542 | 0.0553 | 98.06 |
| t_{1/2} | 13.46 | 13.21 | 101.86 |
| | Test G. Mean | Ref. G. Mean | G Mean Ratio |

Transformed data

| | | | |
|---|---|---|---|
| Cₘₐₓ(ng/ml) | 20.73 | 24.53 | 84.50 |
| AUC₀₋ₜ(ng · hr/ml) | 657.15 | 732.49 | 89.71 |
| AUC_{inf}(ng · hr/ml) | 686.35 | 761.26 | 90.16 |

### Example 2

A 1 mg strength alprazolam extended release tablet of is prepared as follows:

A. Blending

TABLE 3

| | | Amount per | Amount per |
|---|---|---|---|
| Ingredients | % | Unit (mg/tab) | Batch (kg) |
| Alprazolam | 0.2899 | 1.00 | 0.150 |
| Lactose Monohydrate | 76.09 | 262.5 | 39.37 |
| Hypromellose 2208 | 16.50 | 56.93 | 8.540 |
| Hypromellose 2910 | 5.501 | 18.98 | 2.847 |
| Colloidal Silicon Dioxide | 0.500 | 1.725 | 0.2588 |
| Magnesium Stearate | 1 | 3.450 | 0.5175 |
| Coloring Additive | 0.1250 | 0.4313 | 0.06470 |
| Total | 100.00 | 345.0 | 51.75 |

Mix approximately 1 kg of the lactose monohydrate and 0.150 kg of alprazolam for two minutes. Next add 0.06470 kg of the coloring agent to the above mix and continue mixing for 3 minutes. Then mix approximately 4 kg of lactose monohydrate with the above mixture in a 16 quart V-Blender for fifteen (15) minutes. Next place an additional 34.37 kg of lactose monohydrate, the mixture from above, 8.540 kg of hypromellose 2208, 2.847 kg of hypromellose 2910 and 0.2588 kg of colloidal silicon dioxide into a 5 ft³ V-blender and mix for 15 minutes. Then pass the resulting mixture through a comil equipped with a 20 mesh stainless steel screen with no spacer. Charge the screened material into a 5 ft³ V-blender and mix for twenty (20) minutes. Screen 0.5175 kg of magnesium stearate through a #30 mesh screen then add the screened magnesium stearate to the above blend and blend for an additional five minutes.

B. Compression

The blend is then compressed into tablets with tablet weights around 345 mg on a rotary tablet press or an alternative equipment of the same operation principle.

### Example 3

A 2 mg strength alprazolam extended release tablet of is prepared as follows:

A. Blending

TABLE 4

| | | Amount per | Amount per |
|---|---|---|---|
| Ingredients | % | Unit (mg/tab) | Batch (kg) |
| Alprazolam | 0.5797 | 2.00 | 0.300 |
| Lactose Monohydrate | 75.88 | 261.8 | 39.27 |
| Hypromellose 2208 | 16.50 | 56.93 | 8.540 |
| Hypromellose 2910 | 5.501 | 18.98 | 2.847 |
| Colloidal Silicon Dioxide | 0.500 | 1.725 | 0.2588 |
| Magnesium Stearate | 1.000 | 3.450 | 0.5175 |
| Coloring Additive | 0.0300 | 0.1035 | 0.01553 |
| Total | 100.00 | 345.0 | 51.75 |

Mix approximately 1 kg of the lactose monohydrate and 0.300 kg of alprazolam for two minutes. Next add 0.01553 kg of the coloring agent to the above mix and continue mixing for 3 minutes. Then mix approximately 4 kg of lactose monohydrate with the above mixture in a 16 quart V-Blender for fifteen (15) minutes. Next place an additional 34.27 kg of lactose monohydrate, the mixture from above, 8.540 kg of hypromellose 2208, 2.847 kg of hypromellose 2910 and 0.2588 kg of colloidal silicon dioxide into a 5 ft³ V-blender and mix for 15 minutes. Then pass the resulting mixture through a comil equipped with a 20 mesh stainless steel screen with no spacer. Charge the screened material into a 5 ft³ V-blender and mix for twenty (20) minutes. Screen 0.5175 kg of magnesium stearate through a #30 mesh screen then add the screened magnesium stearate to the above blend and blend for an additional five minutes.

B. Compression

The blend is then compressed into tablets with tablet weights around 345 mg on a rotary tablet press or an alternative equipment of the same operation principle.

### Example 4

A 0.5 mg strength alprazolam extended release tablet of is prepared as follows:

A. Blending

TABLE 5

| | | Amount per | Amount per |
|---|---|---|---|
| Ingredients | % | Unit (mg/tab) | Batch (kg) |
| Alprazolam | 0.1449 | 0.500 | 0.0750 |
| Lactose Monohydrate | 76.35 | 263.4 | 39.51 |
| Hypromellose 2208 | 16.50 | 56.93 | 8.540 |
| Hypromellose 2910 | 5.501 | 18.98 | 2.847 |
| Colloidal Silicon Dioxide | 0.500 | 1.725 | 0.2588 |
| Magnesium Stearate | 1.000 | 3.450 | 0.5175 |
| Total | 100.00 | 345.0 | 51.75 |

Mix approximately 1 kg of the lactose monohydrate and 0.150 kg of alprazolam for five minutes. Then mix approximately 4 kg of lactose monohydrate with the above mixture in a 16 quart V-Blender for fifteen (15) minutes. Next place an additional 34.51 kg of lactose monohydrate, the mixture from above, 8.540 kg of hypromellose 2208, 2.847 kg of hypromellose 2910 and 0.2588 kg of colloidal silicon dioxide into a 5 ft³ V-blender and mix for 15 minutes. Then pass the resulting mixture through a comil equipped with a 20 mesh stainless steel screen with no spacer. Charge the screened material into a 5 ft³ V-blender and mix for twenty (20) minutes. Screen 0.5175 kg of magnesium stearate through a #30 mesh screen then add the screened magnesium stearate to the above blend and blend for an additional five minutes.

B. Compression

The blend is then compressed into tablets with tablet weights around 345 mg on a rotary tablet press or an alternative equipment of the same operation principle.

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof that do not depart from the spirit and scope of the invention.

The above-mentioned patents and publications are incorporated herein by reference in their entirety.

We claim:

1. A directly compressible extended release pharmaceutical dosage form comprising a slightly soluble to insoluble benzodiazepine drug and at least two high viscosity pharmaceutically acceptable polymers, wherein said first high viscosity polymer and said second high viscosity polymer are present in a ratio of from about 4:1 to about 2:1.

2. A pharmaceutical dosage form according to claim 1, wherein said slightly soluble to insoluble benzodiazepine drug is alprazolam, bromazepam, chlordiazepoxide, clobazam, clonazepam, clorazepate, diazepam, flunitrazepam, flurazepam, loprazolam, lormetazpam, nitrazepam, oxazepam, and tamazepam.

3. A pharmaceutical dosage form according to claim 1 comprising:
(a) alprazolam;
(b) a filler;
(c) a first high viscosity polymer;
(d) a second high viscosity polymer;
(e) optionally a glidant; and
(f) optionally a lubricant.

4. The pharmaceutical dosage form according to claim 3, wherein the filler is lactose monohydrate.

5. The pharmaceutical dosage form according to claim 3, wherein the glidant is colloidal silicon dioxide.

6. The pharmaceutical dosage form according to claim 3, wherein the first polymer is hypromellose 2208.

7. The pharmaceutical dosage form according to claim 3, wherein the first polymer is hypromellose 2910.

8. The pharmaceutical dosage form according to claim 3, wherein the lubricant is magnesium stearate.

9. A pharmaceutical dosage form according to claim 1 comprising:
(a) 0.01-5% of alprazolam;
(b) 50-99% of a filler;
(c) 1-30% of said first high viscosity polymer;
(d) 1-25% of said second high viscosity polymer;
(e) optionally 0-5% of a glidant; and
(f) optionally 0-10% of a lubricant.

10. A pharmaceutical dosage form according to claim 1 comprising:
(a) 0.1-2.5% of alprazolam;
(b) 65-85% of a filler;
(c) 2-25% of said first high viscosity polymer;
(d) 2-20% of said second high viscosity polymer;
(e) optionally 0-2% of a glidant; and
(f) optionally 0-3% of a lubricant.

11. A directly compressible extended release pharmaceutical dosage form comprising alprazolam and at least two high viscosity pharmaceutically acceptable polymers, wherein the total weight of said first high viscosity polymer and said second high viscosity polymer comprises from about 18% to about 25% of the total dosage form.

12. A pharmaceutical dosage form according to claim 11 comprising:
(a) alprazolam;
(b) a filler;
(c) a first high viscosity polymer;
(d) a second high viscosity polymer;
(e) optionally a glidant; and
(f) optionally a lubricant.

13. A pharmaceutical dosage form according to claim 12 comprising:
(a) 0.01-5% of alprazolam;
(b) 50-99% of a filler;
(c) 1-30% of said first high viscosity polymer;
(d) 1-25% of said second high viscosity polymer;
(e) optionally 0-5% of a glidant; and
(f) optionally 0-10% of a lubricant.

14. A pharmaceutical dosage form according to claim 11 comprising:
(a) 0.1-2.5% of alprazolam;
(b) 65-85% of a filler;
(c) 2-25% of said first high viscosity polymer;
(d) 2-20% of said second high viscosity polymer;
(e) optionally 0-2% of a glidant; and
(f) optionally 0-3% of a lubricant.

15. The pharmaceutical dosage form according to claim 1 that exhibits a peak plasma level between 6 and 12 hours after administration.

16. The pharmaceutical dosage form according to claim 15 that exhibits a peak plasma level between about 8 hours to about 10 hours after administration.

17. The pharmaceutical dosage form according to claim 1 that exhibits a Cₘₐₓ of less than 50 ng/ml.

18. The pharmaceutical dosage form according to claim 17 that exhibits a Cₘₐₓ of less than 30 ng/ml.

19. The pharmaceutical dosage form according to claim 18 that exhibits a Cₘₐₓ of between 15 ng/ml and 25 ng/ml.
